(19) United States
(12) Patent Application Publication
(10) Pub. No.: US 2010/0273215 A1
(43) Pub. Date: Oct. 28, 2010

(54) Secretion Yield of a Protein of Interest by in vivo Proteolytic Processing of a Multimeric Precursor

(76) Inventors: Anna Cornelia Hendrika Van Hautum, Tilburg (NL); Arjo Lysander De Boer, Tilburg (NL); Jan Bastiaan Bouwstra, Tilburg (NL); Peter Franciscus Theresius Maria Van Asten, Tilburg (NL)

Correspondence Address: MORGAN LEWIS & BOCKIUS LLP, 1111 PENNSYLVANIA AVENUE NW, WASHINGTON, DC, 20004, US

(21) Appl. No.: 12/808,326

(22) PCT Filed: Dec. 17, 2008
(86) PCT No.: PCT/NL08/50808

§ 371 Date: Jun. 15, 2010

(30) Foreign Application Priority Data
Dec. 21, 2007 (EP) 07123978.4

(51) Int. Cl. C07H 21/04; C12N 15/63; C12N 1/19; C12P 21/00

(52) U.S. Cl. 435/69.1; 536/23.1; 536/23.5; 536/23.51; 536/23.52; 536/23.53; 536/23.4; 536/23.2; 435/320.1; 435/254.2; 435/254.23

(57) ABSTRACT

The invention relates to a nucleic acid molecule encoding a multimeric precursor which after transcription is specifically cleaved in vivo to form multiple copies of a protein of interest. The invention further relates to a cell comprising this nucleic acid molecule and a method for producing a protein of interest using this cell.

### FIELD OF THE INVENTION

The invention relates to a nucleic acid molecule encoding a multimeric precursor which after transcription is specifically cleaved in vivo to form multiple copies of a protein of interest. The invention further relates to a cell comprising this nucleic acid molecule and a method for producing a protein of interest using this cell.

### BACKGROUND OF THE INVENTION

Secretion systems have been extensively studied. However, the secretion yield of a protein of interest seems to remain a limiting factor in many secretion systems studied so far. For instance, using the Pichia pastoris expression platform, there are relatively few examples of proteins secreted at more than 10 g/L (Werten et al. (1999). Yeast 15: 1087-1096), while many other proteins are secreted at (much) lower levels (see Multi-Copy Pichia Expression Kit, manual version F, 010302, Invitrogen Corporation, and references therein). The secretion yield of a protein of interest also depends on properties of the expression system. A protein of interest may be secreted at different levels in various hosts (Steinborn G. et al., Microb Cell Fact. 2006 Nov. 14; 5:33.) Several strategies have been developed to try to improve the secretion yield of a protein of interest. For example, regulating regions originating from the host used could be used in the expression construct comprising the nucleic acid molecule encoding the protein of interest to be secreted, In yeasts, strong promoters derived from the gene for glyceraldehyde-3-phosphate dehydrogenase are frequently used, and, in methylotrophic yeasts, the strong promoters derived from the gene for peroxisomal alcohol oxidase are frequently used (e.g. Pichia protocols, Methods in Molecular Biology Volume 103, David R. Higgins and James M. Cregg, eds). The gene encoding the protein of interest could be altered to adopt a codon usage similar to the usage in highly expressed genes in the host organism (Grosjean H et al (1982), Gene, 18: 199-209) Alteration of the gene can also prevent premature termination of transcription and enhance stability of the messenger RNA (Scorer C A et al, (1993), Gene, 136:111-119). Frequently the secretion yield can be increased by increasing the gene copy number (Scorer C A et al, Biotechnology (NY). 1994 February; 12(2):181-4.; Higgins D R, et al, Methods Mol Biol. 1998; 103:41-53).

For several proteins of interest it was reported that the secretion yield could be increased by co-overexpression of genes that encode chaperones or other components of the secretory pathway, such as CNEA1 (Conesa A. et al. (2002), Appl. Environ. Microbiol., 68:846-851, Klabunde et al., FEMS Yeast Res. (2005) Oct. 7), PDI1 (Smith JD., et al. (2004), Biotechnol. Bioeng., 85: 340-350, Klabunde J. et al (2005); Inan et al. Biotechnol., Bioeng. (2006), 93:771-778, Liu S H., et al, (2005), Biochem. Biophys. Res. Commun., 326: 81824 and, Lodi T. et al (2005), Appl. Environ. Microbiol. 71: 4359-4363) KAR2 (Smith J D. et al (2004), Biotechnol. Bioeng., 85: 340-350, Klabunde J. et al. (2005)), SEC4 (Liu S H et al (2005), SSO1 and SSO2 (Toikkanen J H. et al, (2004), Yeast, 21: 1045-1055, Ruohonen L. et al, (1997), Yeast, 13:337-351, and Klabunde J. et al (2005)), ERO1 (Lodi T. et al (2005)), SBH1 (Toikkanen J H. et al (2004), Klabunde J. et al (2005)), PSA1 (Uccelletti D. et al, (2005), FEMS Yeast Res., 5: 735-746) UBI4 (Chen Y. et al, (1994) Biotechnology (N.Y.), 12: 819-823), PSE1 (Chow T Y. et al, (1992), J. Cell. Sci., (Pt3):709-719), and DPM1 (Kruszewska J S., et al, (1999), Appl. Environ. Microbiol. 65:2382-2387).

However, the secretion yield of a protein of interest in many secretion systems is not high enough to enable the use of these systems at an industrial scale. Therefore, there is still a need for alternative and optionally improved secretion systems, which do not have all the drawbacks of existing systems.

### SUMMARY OF THE INVENTION

In our study for methods to improve protein secretion we now have surprisingly found, that the amount of protein secretion can be significantly improved by transforming a host cell with a nucleic acid molecule comprising a motif, said motif being repeated at least twice, said motif comprising at least two elements, said two elements being:
   a) an element encoding a protein of interest, and
   b) an element encoding a cleavage site.

It appeared furthermore advantageous in case the cleavage site is a Kex2 cleavage site. The availability of a Kex1 cleavage site gives further improvements. A host cell can be selected from the list of an eukaryotic cell, such as a yeast cell, a fungal cell, a plant cell, a mammalian cell, an insect cell and the like. Using this method, various proteins of interest can be secreted and for each protein of interest in an amount, which was not possible before. It is even possible to design a secretion process, wherein two, three or more distinct proteins of interest are secreted from one single organism.

### DEFINITIONS

A multimer (i.e. polymer) means that a protein of interest comprises a motif or monomer, which is repeated at least twice in a linear fashion to generate a longer polymer or multimer. Such multimers (or polymer) thus comprise or consist of at least 2, 3, 4, 5, 6, 7, 8, 9 or 10 repeats of a monomer sequence. The monomers or monomer units are preferably repeated without intervening amino acids, although optionally 1, 2, 3, 4, 5, or more linking amino acids may be present between some or all of the monomer units.

A homo-multimer Ma means that a protein of interest comprises a single motif M, which is repeated “a” times. The integer “a” may be between 1 and 20 or more, for example between 2 and 50 or, between 2 and 100 or even more. A hetero-multimer means that a protein of interest comprises several distinct motifs or monomers, that are repeated several times: for example M1aM2bM3cM4d. In this example, a motif M1 is repeated “a” times, M2 “b” times, M3 “c” times, M4 “d” times. The integers a, b, c, and d do not have necessary the same value. They are defined as “a” above. M1, M2, M3 and M4 may be the same or different as long at least two monomers are different from each other.

The terms “protein” “protein of interest” or “polypeptide” or “peptide” or “gene product” are used interchangeably and refer to molecules consisting of a chain of amino acids, without reference to a specific mode of action, size, 3-dimensional structure or origin. An isolated protein is a protein not found in its natural environment, such as a protein purified from a culture medium.

The term “enzyme” refers to a protein that has a specific catalytical activity on its substrate such as but not limited to cleaving a specific peptide sequence, removing a specific peptide sequence, cleaving a specific nucleotide sequence, removing a specific nucleotide sequence and the like. This specific catalytical activity is dependant on the concentration of enzyme concentration, substrate concentration and environmental factors such as temperature, acidity, presence of specific ions and other cofactors.

The terms ‘collagen’, ‘collagen-related’, ‘collagen-derived’ and ‘gelatine’ or ‘gelatine-like’ may be used interchangeable.

“Native” or “natural” or “endogenous” protein or of interest means a protein or protein of interest as produced by the organism it originates from.

“Native” or “natural” collagens or collagenous domains refer to those nucleic acid or amino acid sequences found in nature, e.g. in humans or other mammals having MW's ranging from 5,000 up to more than 400,000 daltons.

A gelatine-like protein means either a gelatine-like protein monomer or a gelatine-like protein multimer.

A gelatine-like protein monomer (or a polymer comprising or consisting of monomers) preferably comprises a substantial number, or consists of, GXY triads, wherein G is Glycine and X and Y are any amino acid. A substantial number of GXY triads refers to at least about 50%, more preferably at least 60%, 70%, 80%, 90% or most preferably 100% of amino acid triplets of a whole gelatin-like protein monomer being GXY, especially consecutive GXY triplets. The N- and/or C-terminal end of a monomer and/or polymer may comprise other amino acids, which need not be GXY triplets. Also, the molecular weight of the monomer is preferably at least about 1 kDa (calculated molecular weight), at least about 2, 3, 4, 5, 6, 7, 8, 9 10 or more for example 15, 20, 25, 30 and even 40, 50, 60, 70, 80, 90, or 100 and even higher.

A “fragment” is a part of a longer nucleic acid or polypeptide molecule, which comprises or consist of e.g. at least 10, 15, 20, 25, 30, 50, 100, 200, 500 or more consecutive nucleotides or amino acid residues of a longer molecule. Preferably, a fragment comprises or consists of less than 1000, 800, 600, 500, 300, 200, 100, 50, 30 or less consecutive nucleotides or amino acid residues of a longer molecule.

“Variants” refer to sequences which differ from a natural or native sequence by one or more amino acid insertions, deletions or replacements and are “substantially identical” to a native sequence as defined below.

The term “identity”, “substantially identical”, “substantial identity” or “essentially similar” or “essential similarity” means that two polypeptides, when aligned pairwise using the Smith-Waterman algorithm with default parameters, comprise at least 60%, 70%, 80%, more preferably at least 90%, 95%, 96% or 97%, more preferably at least 98%, 99% or more amino acid sequence identity. Preferably, the alignment is carried out using the whole coding sequence identified by its SEQ ID NO herein. Sequence alignments and scores for percentage sequence identity may be determined using computer programs, such as the GCG Wisconsin Package, Version 10.3, available from Accelrys Inc., 9685 Scranton Road, San Diego, Calif. 92121-3752 USA or using in EmbossWIN (e.g. version 2.10.0). For comparing sequence identity between two sequences, it is preferred that local alignment algorithms are used, such as the Smith Waterman algorithm (Smith T F, Waterman M S (1981) J. Mol. Biol 147(1); 195-7), used e.g. in the EmbossWIN program “water”. Default parameters are gap opening penalty 10.0 and gap extension penalty 0.5, using the Blosum62 substitution matrix for proteins (Henikoff & Henikoff, 1992, PNAS 89, 915-919).

As used herein, the term “operably linked” refers to a linkage of elements (nucleic acid or protein or peptide) in a functional relationship. An element is “operably linked” when it is placed into a functional relationship with another element. For instance, a promoter or enhancer is operably linked to a coding sequence if it affects the transcription of the coding sequence. Operably linked means that the elements being linked are typically contiguous and, where necessary to join two protein coding regions, contiguous and in reading frame.

Expression will be understood to include any step involved in the production of a protein including, but not limited to transcription, post-transcriptional modification, translation, post-translational modification, secretion and the like.

Overexpression will be understood as an increase in the level of expression of an endogenous protein by a modification or multiple modifications to a host cell by any method known in the art. The increase of the level of expression is defined as the increase of the messenger RNA level encoding an endogenous protein by at least 10% as compared to a non-modified host cell. Messenger RNA level may be assessed by Northern blotting or arrays. In case of a protein which is not endogenously expressed in a host cell, if such protein is expressed in said host cell by any method known to the skilled person, preferably by means of recombinant molecular biology technique, one will preferably speak of expression of said protein in said host cell. In this case, expression will lean any detectable amount of mRNA encoding said protein in said host cell.

Nucleic acid construct is defined as a nucleid acid molecule, which is isolated from a naturally occurring gene or which has been modified to comprise segments of nucleic acid which are isolated, synthesised, combined or juxtaposed in a manner which would not otherwise exist in nature.

In addition, reference to an element by the indefinite article “a” or “an” does not exclude the possibility that more than one of the element is present, unless the context clearly requires that there be one and only one of the elements. The indefinite article “a” or “an” thus usually means “at least one”. The term “comprising” is to be interpreted as specifying the presence of the stated parts, steps or components, but does not exclude the presence of one or more additional parts, steps or components. In addition the verb “to consist” may be replaced by “to consist essentially of” meaning that a nucleic acid molecule, a nucleic acid construct, a cell as defined herein may comprise additional component(s) than the ones specifically identified, said additional component(s) not altering the unique characteristic of the invention.

The word “approximately” or “about” when used in association with a numerical value (approximately 10, about 10) preferably means that the value may be the given value of 10 more or less 5% of the value.

All patent and literature references cited in the present specification are hereby incorporated by reference in their entirety.

### DESCRIPTION OF THE FIGURES

FIG. 1. Design of the building block of the multimeric precursor. The desired protein, in this case a gelatin sequence is flanked by an amino acid motif (KREA) indicated in bold and underlined Multimer construction is facilitated by the DraIII and PflmI restriction sites (marked in italics). Binding sites for primers B1-F and B1-R are underlined.

FIG. 2. Plasmid map of pPICZ-B1 with relevant restriction sites.

FIG. 3. The gene for the multimeric precursor B4 (tetrameric precursor). The desired (mature) gelatin sequence is flanked by an amino acid motif (KREA) indicated in bold and underlined. The residues indicated in italics (gpagepg) form an intervening sequence, which is introduced to facilitate multimer construction, using the sites DraIII and PflMI.

FIG. 4. A. SDS-PAGE analysis of culture supernatants from Pichia pastoris strains with a single integrated copy of pB1 (lane 1&2), pB2 (lane 3&4), pB4 (lane 5&6) and pB8 (lane 7&8). M: Low Molecular Weight Marker (Amersham). B SDS-PAGE analysis of culture supernatants from a Pichia pastoris strain with a single integrated copy of pB8 (lane 1) and a strain with a single integrated copy of pB8 that overexpresses the KEX2 gene.

### DETAILED DESCRIPTION OF THE INVENTION

In our search for a method to improve the secretion yield of a protein of interest by a certain micro-organism, we have found that the secretion yield of a protein (expressed in gram per liter) depends on its size. In particular, in the case of a gelatine-like protein, we have found that a gelatine-like protein of higher molecular weight is secreted at higher levels than smaller molecular weight gelatine-like protein. In our further search we sought a novel approach to increase the secreted yield of a protein of interest.

### Nucleic Acid Molecule

In a first aspect, the invention provides a nucleic acid molecule comprising a motif, said motif being repeated at least twice, said motif comprising at least two elements, said at least two elements being:
a) an element encoding a protein of interest, and
b) an element encoding a cleavage site.

An Element Encoding a Protein of Interest (Element a))

A protein of interest may be any protein that can be used in an industrial application such as cosmetic industry, food or feed industry, detergent industry. The protein might be an active ingredient, which may be used as a medicament to prevent, treat, delay any type of disease or condition. A medicament may be for the treatment of pain, cancer, a cardiovascular disease, myocardial repair, angiogenesis, bone repair and regeneration, wound treatment, neural stimulation/therapy or diabetics. Examples of a protein of interest include: a cytokine, an interleukin (IL-2, 4, 5, 6, 12 and the like), an alpha-, beta- and gamma-interferon, a colony stimulating factor (GM-CSF, C-CSF, M-CSF), a chemokine, a hormone (growth hormone, erythropoietin, insulin and the like), a coagulant and an anticoagulant (hirudin and the like) or an anti-oxidant molecule. Further examples are an antibody, an engineered immunoglobulin-like molecule (camelid derived single domain antibodies such as Nanobodies™ and the like, avidity multimers such as Avimers™ and the like or lipocalin derivatives such as Anticalins® and Duocalins® and the like), a single chain antibody or a humanised antibody, an immune co-stimulatory molecule, an immunomodulatory molecule, a transdominant negative mutant of a target protein, another protein capable of inhibiting a viral, bacterial or parasitic infection and/or its development, a structural protein (albumin, collagen and the like), a gelatine or gelatine-like protein, a fusion protein, an enzyme (trypsin, a ribonuclease, a P450 cytochrome, a lipase, an amylase and the like), a toxin, a conditional toxin, an antigen, a protein capable of inhibiting the initiation or progression of a tumour or a cancer (an inhibitor acting at the level of cell division or of transduction signals, a product of expression of tumor suppressor genes, for example p53 or Rb and the like), a growth factor, a membrane protein, a vasoactive protein and a derivative thereof (such as with an associated reporter group). A protein of interest may also comprises a pro-drug activating enzyme.

In another embodiment, a protein of interest is itself a multimer: homomultimer or heteromultimer as herein defined under the section general definition.

In still another embodiment, a protein of interest which is itself a multimer is a gelatine-like protein as herein defined in the section entitled general definitions.

An Element Encoding a Cleavage Site (Element b))

An element (b) of the motif being present in a nucleic acid sequence of the nucleic acid molecule of the invention, encodes a cleavage site. Such a cleavage site may be any cleavage site known in the art. In this invention, good results were obtained, by using a Kex2 cleavage site. Preferably, a cleavage site is a Kex2 cleavage site.

In a preferred embodiment, a motif present in a nucleic acid molecule of the invention additionally comprises a third element being an intervening sequence.
In another preferred embodiment, a nucleic acid molecule of the invention comprises a motif, said motif being repeated at least twice, said motif comprising at least two elements wherein:
   element a) encodes a protein of interest wherein at least two distinct elements a) are present, each encoding a distinct protein of interest and
   element b) encodes a cleavage site wherein at least two b) elements are present, each encoding a cleavage site, so that a cleavage site is present between each protein of interest.

This nucleic acid molecule is preferred since it will allow the production of a mixture of proteins of interest which mixture can comprise distinct proteins of interest. An example of the beneficial use of this embodiment is the manufacturing of the Hepatitis vaccine. This vaccine comprises several distinct proteins.

In a nucleic acid molecule of this invention, a motif as defined herein is repeated at least twice, but it may be repeated many times, for example from 2 to 100 times or more. For example, a motif may be repeated 3, 4, 5, 6, 7, 8, 9, 10 or 15, 20, 25, 30, or 40, 50, 60, 70, 80, 90, 95 times or more.

In case, a protein of interest itself contains a cleavage site like for example a Kex2-like or Kex2 cleavage site, said protein of interest may also be cleaved as such, which is not desired. In such a case, one could consider to remove a cleavage site from a native sequence encoding a protein of interest. As a result, a multimeric precursor comprising a variant of a native protein of interest will be produced and subsequently, a variant of a native protein of interest will be secreted by a method of this invention. In a preferred embodiment, a native protein of interest does not have an internal cleavage site, more preferably an Kex2 cleavage site.

The expression of a repeated motif in a nucleic acid sequence of the nucleic acid molecule of the invention will result in the expression of a multimeric precursor comprising a protein of interest. Each protein of interest within this multimeric precursor is separated a cleavage site. By the action of a specific cleavage enzyme, an individual protein of interest can be secreted giving a very high yield in fermentation processes for a protein of interest.

As indicated in the previous paragraph, a native protein of interest may already comprise at least one cleavage site. In a nucleic acid molecule of the invention comprising elements a) and b) as defined above, element b) is preferably not present within a protein of interest (element a)) but present upstream and downstream of a protein of interest. If a protein of interest contains at least one cleavage site, this at least one site is preferably removed. The skilled person knows how to specifically remove such sites in a given protein sequence by manipulating a corresponding coding sequence.

A Kex2 cleavage site is a site which is cleavable by a Kex2 or a Kex2-like enzyme. Kex2 is the name of the Saccharomyces cerevisiae enzyme (Kurjan & Herskowitz, (1982) Cell, 30:933-943, Brake A J et al (1983), Mol. Cell. Biol., 3: 1440-1450 and Caplan et al, (1991), J. Bacteriol., 173: 627-635). However, Kex2-like enzymes or Kex2 homologues have been already identified in several eukaryotes including plants (Jiang L et al, (1999), The Plant Journal, 18: 23-32, Fuller R S, et al, (1989), J. Science, 246:482-486 and Bresnata P A et al, (1990), J. Cell. Biol., 111: 2851-2859). We propose the following definition for a Kex2-like enzyme: this is a Golgi-localised protease that specifically cuts proteins after two consecutive basic amino acid residues such as K or R. Therefore, a Kex2 cleavage site is preferably: KK, KR, RR or RK. KR is a more preferred cleavage site. However, some sequences containing a single R residue are also cleaved by Kex2. For instance, Kex2 cleaves after the R residues in the sequence MGPR that occurs in certain collagenous sequences (Werten M W, de Wolf F A. Reduced proteolysis of secreted gelatin and Yps1-mediated alpha-factor leader processing in a Pichia pastoris kex2 disruptant. Appl Environ Microbiol. 2005 May; 71(5):2310-7).

Therefore, a nucleic acid molecule comprising as element b, a nucleic acid molecule encoding any of KK, KR, RR, or RK is encompassed by the present invention also including a nucleic acid molecule encoding a MGPR sequence. The efficiency of cleavage by a Kex2-like enzyme is dependent on the residue that follows the dibasic motif. For instance, in the prototypic Kex2 cleavage site KRX, several amino acids X are tolerated, such as aromatic amino acids, small amino acids and histidine (Multi-Copy Pichia Expression Kit, manual version F, 010302, Invitrogen Corporation). In yeast, very efficient cleavage occurs when the dibasic Kex2 cleavage site is followed by EA, or repeats thereof. These EA repeats are typically removed by the STE13 (STErile 13) gene product (Julius D, Blair L, Brake A, Sprague G, Thorner J. Yeast alpha factor is processed from a larger precursor polypeptide: the essential role of a membrane-bound dipeptidyl aminopeptidase. Cell. 1983 March; 32(3):839-52.). In an embodiment, such EA motif, when located in the context of a Kex2 cleavage site, for example KREA, is named a STE13 cleavage site.

Therefore, in a preferred embodiment, a Kex2 cleavage site is defined by a dibasic motif as defined herein followed by EA or repeats thereof.

In a more preferred embodiment, a nucleic acid molecule is used that encodes a multimeric precursor comprising several copies of a protein of interest, each copy of a protein of interest being separated by a sequence selected from KREA, KREAEA and KREAEAEA. This nucleic acid molecule is introduced in a host cell, preferably a yeast cell, that comprises a Kex2-like protein, a Kex1-like protein and a protein with the same activity as the STE13 gene product.

A nucleic acid molecule of the invention may encode a polypeptide that can be represented with the formula:

(CP)n or (CP)nC wherein P is a Protein of interest as defined herein, C is a dipeptide that comprises a cleavage site preferably a Kex2 cleavage site as defined herein, and n is an integer which is at least 2. In a preferred embodiment, n is at least 3, 4, 5, 6, 7, 8, 9, 10, 11, 12, 13, 14, 15, 16, 17, 18, 19, 20 or 50, 100, 150, 200 or more. Alternatively or in combination with earlier preferred embodiments, the invention relates to a preferred embodiment wherein a leader sequence (L) is present upstream of the first CP motif: L(CP)n or L(CP)nC. A preferred leader sequence is the alpha factor prepro sequence (Brake A J et al, (1983), Mol. Cell. Biol., 3:1440-1450, Kurjan & Herskowitz, (1982), Cell, 30: 933-943).

A nucleic acid molecule according to the invention is particularly advantageous since when translated into a corresponding protein in a host, each Kex2 cleavage site will be recognized and cleaved by a K 2-like protease present in a host cell, resulting in the production of a protein of interest, typically with a C-terminal extension (left over from the Kex2 site), since Kex2 cleaves C-terminally of its recognition site. Optionally a Kex1-like enzyme removes the C-terminal basic residues (left over from the Kex 2 site) (Wagner J C & Wolf D H., (1987), FEBS Letters, 14: 423-426 and Cooper A. & Bussey H., (1989), Mol. Cell. Biol., 9: 2706-2714). For example, cleavage of the sequence LCPCPCPCP by Kex 2 results in the production of LC, PC, PC, PC and P. Further cleavage by Kex1 results in the production of 4 copies of a protein of interest P (and L and C).

Therefore, one single nucleic acid molecule of the invention will lead to the production of n copies of a protein of interest. The yield of a protein of interest to be produced in a fermentation process could therefore be expected to increase in comparison to the yield of a same protein of interest in a same host cell but using a classical nucleic acid molecule (or nucleic acid construct or expression construct), said classical nucleic acid molecule comprising one single copy of a nucleic acid molecule encoding a protein of interest. We indeed found to our surprise, that this theory could be reduced into practice. The skilled person will understand that the expected increase will vary depending on among others the chosen host cell and the chosen protein of interest. For some host cell-protein of interest combinations only a small increase of 2%, 3%, 4%, 5%, 7%, or 10% can be achieved. For other combinations the increase can be more significant like for example at least 20%, 30%, 40%, 50%, 60%, 70%, 80% or 90% or more and for again other combinations the yield can be increased by a factor of 2, 3, 4, 5, 6, 7 8, 9 and even 10 or more. The yield is preferably assessed by a quantitative method. For instance, the amount of a protein of interest in the culture supernatant can be determined by a chromatographic procedure such as HPLC or GPC (gel permeation chromatography) by comparison with a known amount of the same protein. Yields may also be compared by using a specific assay for an activity of a protein of interest, if such an assay is available. If the yield is increased by at least 50%, semi-quantitative methods such as SDS-PAGE or Western blotting may also be used.

In an embodiment, each element a) encoding a protein of interest may be operably linked to each element b) encoding a cleavage site, preferably a Kex2 cleavage site. Alternatively or in combination with other embodiments, the invention relates to another preferred embodiment, wherein a nucleic acid molecule of the invention encodes a multimeric precursor represented by . . . CPCQCRCS, wherein C is a cleavage site like for example a dipeptide comprising a Kex2 cleavage site, P, Q, R, S are four distinct polypeptides of interest. Of course, this is an example, other embodiments of the invention cover a nucleic acid molecule allowing the production of two, three, four, five, six, seven, eight, nine, ten or more distinct proteins of interest. This embodiment of the invention is particularly advantageous for the production of a vaccine, which comprises several peptides or proteins of interest. An example of such a vaccine is a Hepatitis vaccine.

Alternatively or in combination with earlier preferred embodiments, the invention relates to another preferred embodiment, wherein a nucleic acid molecule of the invention encodes a multimeric precursor that may be depicted as follows: (CPCI)n in which C represents a cleavage site like for example a dipeptide comprising a Kex2 cleavage site, P represents a protein of interest, I represents an intervening sequence and n is an integer which is at least 2. In a preferred embodiment, n is at least 3, 4, 5, 6, 7, 8, 9, 10, 11, 12, 13, 14, 15, 16, 17, 18, 19, 20, 50, 100, 200 or more. More preferably, a leader sequence is present upstream of the first motif CPCI: L(CPCI)n. A preferred leader has already been defined herein.

An intervening sequence usually comprises 3, 4, 5, 6, 7, 8, 9, amino acids. In one embodiment, an intervening sequence comprises for example 7 amino acids. An intervening sequence can also comprise an additional cleavage site like for example a Kex2 cleavage site. An intervening sequence may represent a practical result or side result of the design or construction of a nucleic acid molecule of the invention. A preferred intervening sequence is formed by construction of a nucleotide of the invention using restriction endonucleases.

The preparation of a nucleic acid molecule of the invention is carried out using molecular biology techniques known to the skilled person (J. Sambrook et al Molecular Cloning: A Laboratory Manual, 2001, 3^{rd} edition, Cold Spring Harbor laboratory).

### Nucleic Acid Construct or Expression Vector

In a further aspect, there is provided a nucleic acid construct or expression vector comprising a nucleic acid molecule as defined in the previous section.

Optionally, a nucleic acid molecule present in a nucleic acid construct is operably linked to one or more control sequences, which direct the production of an encoded protein in a suitable expression host.

Control sequence is defined herein to include all components, which are necessary or advantageous for the expression of a protein of interest. At a minimum, the control sequences include a promoter and trancriptional and translational stop signals.

The invention also relates to an expression vector comprising a nucleic acid construct of the invention. Preferably, an expression vector comprises a nucleic acid molecule of the invention, which is operably linked to one or more control sequences, which direct the production of an encoded protein of interest in a suitable expression host. At a minimum control sequences include a promoter and transcriptional and translational stop signals. An expression vector may be seen as a recombinant expression vector. An expression vector may be any vector (e.g. plasmic, virus), which can be conveniently subjected to recombinant DNA procedures and can bring about the expression of a nucleic acid sequence encoding a recombinant protein of interest. Depending on the identity of the host wherein this expression vector will be introduced and on the origin of the nucleic acid sequence of the invention, the skilled person will know how to choose the most suited expression vector and control sequences.

### Host Cell

In yet a further aspect, there is provided a host cell or host or cell comprising a nucleic acid construct or expression vector as defined in the previous section.

Preferably, a host cell is an eukaryotic cell such as a yeast cell, a fungal cell, a plant cell, a mammalian cell, an insect cell and the like. It is to be noted that the invention could be applied in at least any cell expressing a functional Kex2-like and preferably also a Kex1-like enzyme. Preferred mammalian cells are human cells. Yeast cells can be selected from Hansenula, Trichoderma, Aspergillus, Penicillium, Saccharomyces, Kluyveromyces, Neurospora, Arxula or Pichia. Fungal and yeast cells are preferred to bacteria as they are less susceptible to improper expression of repetitive sequences. Yeast cells are even more preferred. Methylotrophic yeast hosts are most preferred. Examples of methylotrophic yeasts include strains belonging to Hansenula or Pichia species. Preferred species include Hansenula polymorpha and Pichia pastoris. More preferably, a host will not have a high level of a protease and/or a proteolytic enzyme that could have attacked or degraded a protein of interest when expressed. Even more preferably, a host has been modified to be deficient in one or more proteases and/or proteolytic enzymes and/or other undesirable enzymes. In this context, a protease is preferably not a Kex2-like or a Kex1-like enzyme. Examples of undesirable enzymes are proteinase A or B. In this respect, Pichia or Hansenula offers an example of a very suitable expression system. Use of Pichia pastoris as an expression system for gelatins is disclosed in EP-A-0926543 and EP-A-1014176. The selection of a suitable host cell from known industrial enzyme producing fungal host cells specifically yeast cells on the basis of the required parameters described herein rendering a host cell suitable for expression of a protein of interest suitable to be used according to the invention in combination with knowledge regarding the host cells and the sequence to be expressed will be possible by a person skilled in the art.

In a preferred embodiment, a host cell comprises or expresses and/or overexpresses a Kex2-like enzyme or an enzyme having Kex2-like processing activity or enhanced Kex2-like processing activity. More preferably, a host cell expresses a functional endogenous (i.e. native) Kex2-like enzyme.

Alternatively or in combination with previous preferred embodiment, a host cell is engineered in order to (over)express a Kex2-like enzyme and/or engineered in order to exhibit an enhanced Kex2-like processing activity. In this embodiment, a host cell may already express a functional endogenous Kex2-like enzyme. An endogenous Kex2-like enzyme and/or a non native Kex2-like enzyme may be overexpressed in a host cell. The host cell used is said to express a functional Kex2-like enzyme or to possess or comprise a Kex2-like processing activity if a cell is able to specifically cleave a Kex2 cleavage site in vivo or in vitro, said site being as earlier defined herein to at least 50%, at least 60%, at least 70%, at least 80%, at least 90%, preferably at least 95%, at least 99% or 100%. The ability of a given Kex2-like enzyme to cleave a Kex2 cleavage site in vivo is preferably assessed by transforming a cell expressing said Kex2-like enzyme with a nucleic acid construct comprising a nucleic acid sequence encoding a polypeptide comprising a Kex2 or a Kex2-like site, culturing transformed cells and assessing as a percentage the functionality of the Kex2-like enzyme of the cell. The in vitro assessment is preferably carried out on said Kex2-like enzyme to be tested which is incubated with a polypeptide comprising a Kex2 cleavage site as earlier defined herein.

Alternatively, the functionality of a Kex-2 like enzyme is assessed by comparison with the activity of the Kex-2 activity of the Kex2 enzyme from Saccharomyces cerevisae or Pichia pastoris. In this embodiment, a Kex2-like enzyme is preferably said functional or said to possess or comprise a Kex2-like processing activity if its capacity to cleave a given Kex2 cleavage site is at least 50% of the capacity of the Kex2 of Saccharomyces cerevisiae or Pichia pastoris to cleave said same cleavage site. Preferably, the cleavage capacity is of at least 60%, 70%, 80%, 90% or 100% or higher. In this embodiment, the assessment may be carried out in vitro or in vivo as earlier defined herein.

A Kex2-like processing activity is preferably said to be enhanced when it is enhanced of at least 5% in an engineered given cell measured using any of the assays given above by comparison with the same activity in the cell it originates from. Alternatively, the activity is assessed in in vitro as earlier defined herein. More preferably, enhanced means, an enhancement of at least 10%, 15%, 20%, 25%, 30%, 35%, 40%, 45%, 50%, 60%, 70%, 80%, 90%, 100%, 150%, 200% or more.

The presence of a cleaved product as a result of the presence of a Kex2-like activity may be assessed by mass spectrometry.

Even more preferably, a host cell further expresses or comprises and/or overexpresses a Kex1-like enzyme or an Kex-1 like having Kex1-like processing activity or enhanced Kex1-like processing activity. Even more preferably, a host cell expresses a functional endogenous Kex1-like enzyme to yield a protein of interest without any additional C-terminal residues (left over from a Kex2 site) as already defined herein. Kex1 is the name of the enzyme as identified in Saccharomyces cerevisiae (Wagner J C, et al (1987), Febs Lett., 221: 423-436). As for Kex2, several eukaryotes homologues of Kex1 have already been identified. In another even more preferred embodiment, a host cell is engineered in order to overexpress a Kex1-like enzyme and/or engineered in order to exhibit an enhanced a Kex1-like processing activity, preferably a Kex1 cleavage site. In this embodiment, a host cell may already express a functional endogenous Kex1-like enzyme. An endogenous Kex1-like enzyme and/or a non native Kex1-like enzyme may be overexpressed in a host cell. A host cell used is said to express a functional Kex1-like enzyme or to possess a Kex1-like processing activity if a cell is able to specifically cleave a Kex1 cleavage site in vivo or in vitro, said site being as earlier defined herein to at least 50%, at least 60%, at least 70%, at least 80%, at least 90%, preferably at least 95%, at least 99% or 100%. The ability of a given Kex1-like enzyme to cleave a Kex1 cleavage site in vivo is preferably assessed by transforming a cell expressing said Kex1-like enzyme with a nucleic acid construct comprising a nucleic acid sequence encoding a polypeptide comprising a Kex1 or a Kex1-like site, culturing transformed cells and assessing as a percentage the functionality of the Kex1-like enzyme of the cell. The in vitro assessment is preferably carried out on said Kex1-like enzyme to be tested which is incubated with a polypeptide comprising a Kex1 cleavage site as earlier defined herein.

Alternatively, the functionality of a Kex-1 like enzyme is assessed by comparison with the activity of the Kex-1 activity of the Kex1 enzyme from Saccharomyces cerevisae or Pichia pastoris. In this embodiment, a Kex1-like enzyme is preferably said functional or said to possess or comprise a Kex1-like processing activity if its capacity to cleave a given Kex1 cleavage site is at least 50% of the capacity of the Kex1 of Saccharomyces cerevisiae or Pichia pastoris to cleave said same cleavage site. Preferably, the cleavage capacity is of at least 60%, 70%, 80%, 90% or 100% or higher. In this embodiment, the assessment may be carried out in vitro or in vivo as earlier defined herein.

A Kex1-like processing activity is preferably said to be enhanced when it is enhanced of at least 5% in an engineered given cell measured using any of the assays given above by comparison with the same activity in the cell it originates from. Alternatively, the activity is assessed in in vitro as earlier defined herein. More preferably, enhanced means, an enhancement of at least 10%, 15%, 20%, 25%, 30%, 35%, 40%, 45%, 50%, 60%, 70%, 80%, 90%, 100%, 150%, 200% or more.

The presence of a cleaved product as a result of the presence of a Kex1-like activity ay be assessed by mass spectrometry.

Even more preferably, a host cell further expresses or comprises and/or overexpresses a STE13 gene-like product or a STE13 gene product having the same activity or enhanced activity as a STE13 gene product. Even more preferably, a host cell expresses a functional endogenous STE13 gene-like product to yield a protein of interest without any additional EA motif (left over from a Kex2 site) as already defined herein. Ste13 is the name of the enzyme of Saccharomyces cerevisiae as identified in Julius et al (Julius D. et al, (1983), Cell, 32: 839-852). As for Kex2, several eukaryotes homologues of Ste13 have already been identified. In another even more preferred embodiment, a host cell is engineered in order to overexpress a STE13 gene-like product and/or engineered in order to exhibit an enhanced STE13 gene product processing activity. In this embodiment, a host cell may already express a functional endogenous STE13 gene product. An endogenous STE13 gene product and/or a non native STE13 gene product may be overexpressed in a host cell. The host cell used is said to express a functional STE13 gene product or to possess a processing activity of a STE13 gene product if a cell is able to specifically cleave a STE13 cleavage site in vivo or in vitro, said site being as earlier defined herein to at least 50%, at least 60%, at least 70%, at least 80%, at least 90%, preferably at least 95%, at least 99% or 100%. The ability of a given STE13-like enzyme to cleave a STE13 cleavage site in vivo is preferably assessed by transforming a cell expressing said STE13-like enzyme with a nucleic acid construct comprising a nucleic acid sequence encoding a polypeptide comprising a STE13 or a STE13-like site, culturing transformed cells and assessing as a percentage the functionality of the STE13-like enzyme of the cell. The in vitro assessment is preferably carried out on said STE13-like enzyme to be tested which is incubated with a polypeptide comprising a STE13 cleavage site as earlier defined herein.

Alternatively, the functionality of a STE13 like enzyme is assessed by comparison with the activity of the STE13 activity of the STE13 enzyme from Saccharomyces cerevisae or Pichia pastoris. In this embodiment, a STE13-like enzyme is preferably said functional or said to possess or comprise a STE13-like processing activity if its capacity to cleave a given STE13 cleavage site is at least 50% of the capacity of the STE13 of Saccharomyces cerevisiae or Pichia pastoris to cleave said same cleavage site. Preferably, the cleavage capacity is of at least 60%, 70%, 80%, 90% or 100% or higher. In this embodiment, the assessment may be carried out in vitro or in vivo as earlier defined herein.

A STE13-like processing activity is preferably said to be enhanced when it is enhanced of at least 5% in an engineered given cell measured using any of the assays given above by comparison with the same activity in the cell it originates from. Alternatively, the activity is assessed in in vitro as earlier defined herein. More preferably, enhanced means, an enhancement of at least 10%, 15%, 20%, 25%, 30%, 35%, 40%, 45%, 50%, 60%, 70%, 80%, 90%, 100%, 150%, 200% or more.

The presence of a cleaved product as a result of the presence of a STE13-like activity may be assessed by mass spectrometry.

In a preferred embodiment, a cell comprises, expresses and/or overexpresses a Kex2-like enzyme or an enzyme having Kex2-like processing activity or enhanced Kex2-like processing activity and optionally
further expresses or comprises and/or overexpresses a Kex1-like enzyme or a Kex-1 like having Kex1-like processing activity or enhanced Kex1-like processing activity and/or
further expresses or comprises and/or overexpresses a STE13 gene-like product or a STE13 gene product having the same activity or enhanced activity as a STE13 gene product.

Alternatively or in combination with earlier mentioned embodiments, a host cell is transformed with a nucleic acid construct comprising a nucleic acid molecule encoding a Kex2-like and optionally a Kex1-like enzyme and/or a STE13 gene-like product. A nucleic acid sequence encoding a Kex1 enzyme is given as SEQ ID NO:1. A nucleic acid sequence encoding a Kex2 enzyme is given as SEQ ID NO:2. A corresponding encoded Kex2 is given as SEQ ID NO:3. A corresponding encoded Kex1 is given as SEQ ID NO:4. A nucleic acid sequence encoding a STE13 gene product is given as SEQ ID NO:5. A corresponding STE13 gene product is given as SEQ ID NO:6.

A nucleic acid sequence encoding a Kex2-like enzyme which is preferably used in this invention has at least 60% identity with SEQ ID NO:2. More preferably, at least 70%, 75%, 80%, 85%, 90%, 95%, 97%, 99% or more.

A nucleic acid sequence encoding a Kex1-like enzyme which is preferably used in this invention has at least 60% identity with SEQ ID NO:1. More preferably, at least 70%, 75%, 80%, 85%, 90%, 95%, 97%, 99% or more.

A nucleic acid sequence encoding a STE13 gene product which is preferably used in this invention has at least 60% identity with SEQ ID NO:5. More preferably, at least 70%, 75%, 80%, 85%, 90%, 95%, 97%, 99% or more.

The skilled person will know that depending on the identity of the host cell chosen, he will choose the most appropriate sequence encoding a Kex2, optionally Kex1 enzyme and/or STE13 gene product to ensure expression of a Kex2, optionally Kex1 enzyme and/or STE13 gene product.

### Production Method

In yet a further aspect, there is provided a method for the production of a protein of interest using a cell as defined in the former section. In this method, preferably a host cell as defined in the previous section is cultured under suitable conditions leading to expression of a protein of interest. Although not preferred optionally the protein of interest can also be recovered from a host cell.

A preferred method for producing a protein of interest according to present invention comprises:
preparing an expression vector comprising a nucleic acid molecule as defined in the section “nucleic acid molecule”
expressing said nucleic acid molecule in a host, preferably a yeast, more preferably a methylotrophic yeast,
culturing said yeast under suitable fermentation conditions to allow expression of said nucleic acid molecule and preferably secretion of said protein of interest;
purifying said protein of interest from the culture.

A protein of interest, like for example a gelatine-like protein may be produced by recombinant methods as disclosed in EP-A-0926543, EP-A-1014176 or WO01/34646. Also for enablement of the production and purification of a protein of interest, like for example a gelatine-like protein reference is made to the examples in EP-A-0926543 and EP-A-1014176 wherein Pichia pastoris is used as host cell.

By using a method for the production of a protein of interest of this invention such recombinantly made proteins of interest can now be made on an industrial scale in an economical way. These proteins can be used in various applications depending on their identity.

The following examples are offered for illustrative purposes only, and are not intended to limit the scope of the present invention in any way.

### EXAMPLES

In this example, a gelatin-like protein is taken as an example of a protein of interest. We created a large molecule with several copies of a desired gene encoding a gelatin-like protein. Upon expression of this large molecule, the encoded polypeptides were produced and subsequently processed in vivo by proteases to release multiple copies of the desired gelatin-like protein.

A desired gelatin-like protein fragment was separated by a dibasic cleavage site (in particular, KR followed by EA). The yeast protease Kex2 cleaves proteins immediately C-terminally of the dibasic amino acid motif. In Pichia pastoris, also Yps1 also cuts C-terminally of this dibasic amino acid motif. This Yps1 site behaves similar in other micro-organisms.

Another yeast protease, Kex1, removes the basic amino acid residues that are left at the C-terminus after cleavage by Kex2. Kex1 and Kex2 are proteases of the yeast Golgi apparatus. Yps1 is located at the plasma membrane. The STE13 gene product removes EA dipeptides left N-terminally of fragments after cleavage by Kex2. All four proteolytic enzymes act on proteins that pass through the secretory pathway. Thus, a large gelatine-like protein precursor mentioned above will be processed intracellularly during secretion and the desired, small gelatine-like protein fragments will be secreted into the medium.

### Design of the Precursor

As the basis for this test, the P monomer or Polar gelatin (see Werten M W, Wisselink W H, Jansen-van den Bosch T J, de Bruin EC, de Wolf F A. Secreted production of a custom-designed, highly hydrophilic gelatin in Pichia pastoris. Protein Eng. 2001 June; 14(6):447-54.) was chosen, because of its high stability towards chemical and/or proteolytic degradation. Use of a “stable” gelatine-like protein should facilitate interpretation of the results.

The precursor comprises the α mating factor secretion signal, followed by a dibasic cleavage site and several copies of the gelatin-like protein and intervening sequences are present, separated by the same dibasic cleavage site and an intervening sequence (in particular, KR followed by EA). The yeast protease Kex2 cleaves proteins immediately C-terminally of the dibasic amino acid motif. In Pichia pastoris, also Yps1 also cuts C-terminally of this dibasic amino acid motif. Yps1 may behave similar in other micro-organisms.

Another yeast protease, Kex1, removes the basic amino acid residues that are left at the C-terminus after cleavage by Kex2. Kex1 and Kex2 are proteases of the yeast Golgi apparatus. Yps1 is located at the plasma membrane. The STE13 gene product removes EA dipeptides left N-terminally of fragments after cleavage by Kex2. All four proteolytic enzymes act on proteins that pass through the secretory pathway.

Thus, the large gelatine-like protein precursor mentioned above will be processed intracellularly during secretion and the desired, small gelatin-like protein fragments will be secreted into the medium.

### Method

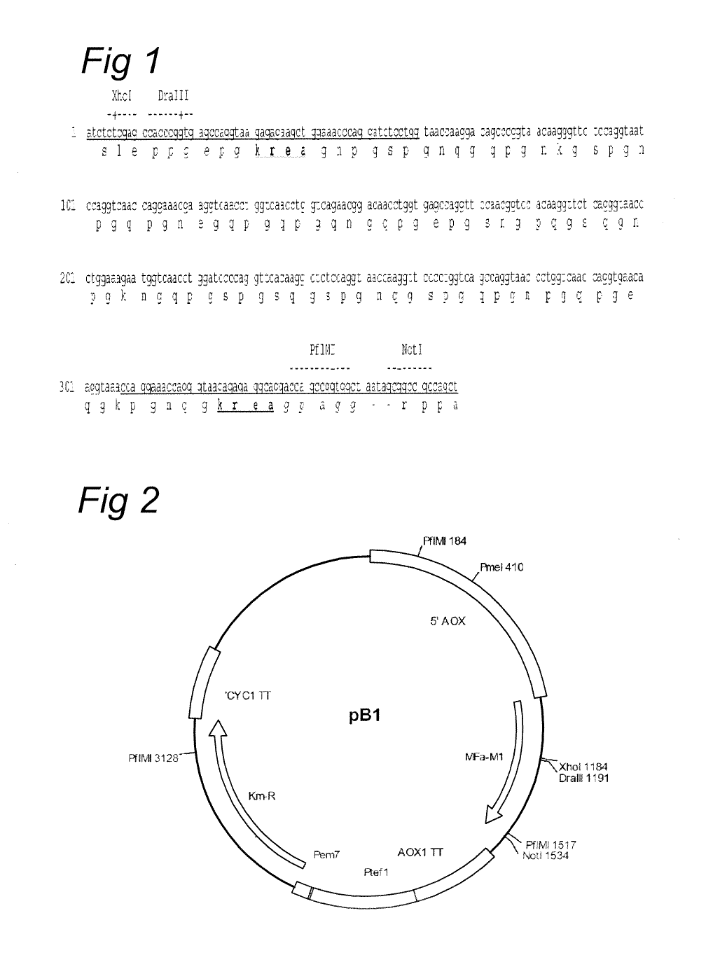

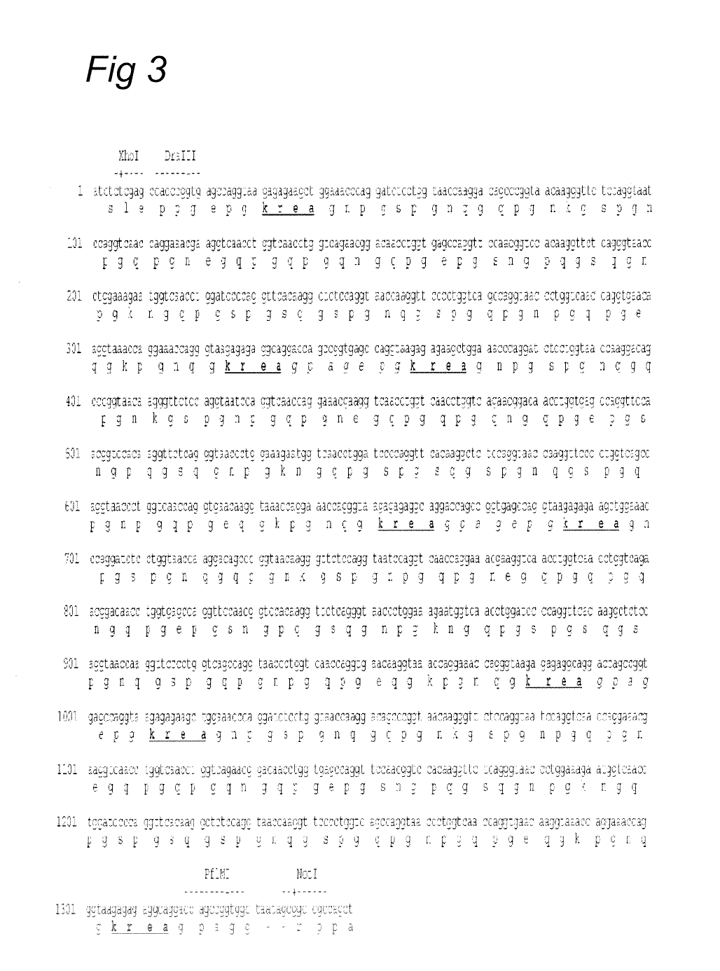

A building block for the construction of nucleic acids for the invention was created by amplification of the gene for the P monomer from pPIC-P (Werten M W, Wisselink W H, Jansen-van den Bosch T J, de Bruin E C, de Wolf F A. Secreted production of a custom-designed, highly hydrophilic gelatin in Pichia pastoris Protein Eng. 2001 June; 14(6):447-54) using primers B1-F and B1-R. The structure and sequence of this building block are represented in FIG. 1. The resulting PCR product was cloned into pPICKα A, using the restriction sites XhoI and NotI, resulting in pB1 (FIG. 2). pPICKαA is a modification of pPICZα A (from Invitrogen) by replacing the coding sequence of the zeocin gene of the latter with the coding sequence of the kanamycin gene from pPIC9K (Invitrogen). The kanamycin resistance gene in pPICKα A confers resistance to kanamycin in E. coli and resistance to G418 or Geneticin in yeast. Multimers (B2, B4, B8) were created essentially as described by Werten et al, 2001 (using the facts that some DraIII sites and PflMI sites have compatible ends after digestion, and that upon ligation neither of the original sites is formed back). The cloning steps are summarized in Table 1. The structure of the large molecule encoding a polypeptide that comprises multiple copies of the gelatin of interest and of the intervening sequence is illustrated in FIG. 3 for a gene that encodes a polypeptide that comprises 4 copies of the desired gelatin.

The plasmids pB1, pB2, pB4 and pB8 were linearized with PmeI and introduced in Pichia pastoris X-33. Single copy transformants were obtained by selection on YPD plates containing 0.5 mg/ml geneticin. The copy number was confirmed by Southern blot analysis as follows. The AOX1 promoter of wild type Pichia pastoris is located on an Acc65 I fragment of about 2.2 kb. Upon integration of a in the AOX1 promoter, this fragment will increase with the size of the plasmid, because the plasmids all lack an Acc65 I site.

Genomic DNA from several transformants was isolated, digested with Acc65 I, electrophoresed and blotted on a membrane. The membrane was probed with a fragment (about 1.2 kb) that contains the AOX1 promoter. Fragments of about 6.5 kb, 6.8 kb, 7.5 kb an 8.8 kb as expected for integration of respectively pB1, pB2, pB4 and pB8 as a single copy in the AOX1 promoter were observed.

TABLE 1
Cloning strategy for creating multimers.

| | Digest | Expected | Digest with | Expected | |
|---|---|---|---|---|---|
| | with PmeI | fragment | PmeI and | fragment | |
| Construction of | and PflMI | sizes | DraIII | sizes | Ligate fragments |
| pB2 | pB1 | 223 | pB1 | 783 | 1110 and 3501 |
| | | 1110 | | 3501 | |
| | | 1340 | | | |
| | | 1611 | | | |
| pB4 | pB2 | 223 | pB2 | 783 | 1437 and 3828 |
| | | 1340 | | 3828 | |
| | | 1437 | | | |
| | | 1611 | | | |
| pB8 | pB4 | 223 | pB4 | 783 | 2091 and 4482 |
| | | 1340 | | 4482 | |
| | | 1611 | | | |
| | | 2091 | | | |

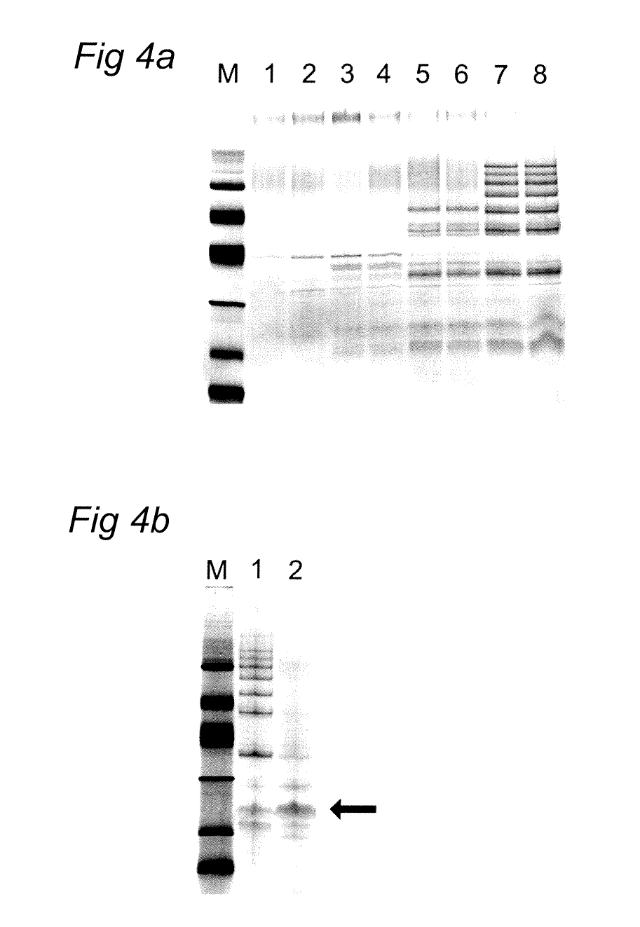

Small scale expression studies of two single copy transformants of each of the plasmids were performed following the suggestions in the manual of the Multi-Copy Pichia Expression Kit, manual version F, 010302, Invitrogen Corporation. Fermentations were performed using the modified Pichia strains as described in EP-A-0926543 and EP-A 1014176. Culture supernatants were analyzed by SDS-PAGE (FIG. 4 A).

For the multimers, both partially and fully processed forms were observed, indicating incomplete processing by the KEX2 enzyme. The total amount of gelatin-like protein secreted increased with the size of the precursor.

In order to improve processing by KEX2, the KEX2 coding sequence was cloned in pGAPZ A (Invitrogen). The resulting plasmid was linearized with Hpa I to promote integration in the GAP promoter and transformed into a Pichia pastoris that contained a single integrated copy of pB8. As can be seen from FIG. 4B, overexpression of the KEX2 gene resulted in complete processing of the B8 precursor. The amount of B formed with the pB8 plasmid with Kex2 overexpression (FIG. 4B, lane 2) is much higher than the amount of B produced from the pB1 plasmid (FIG. 4A, lanes 1&2). Thus, we surprisingly found that culture supernatants from Pichia pastoris strains with a single integrated copy of the plasmid pB2, pB4 or pB8 produced much more of the gelatin of interest, B, than the control strain with a single integrated copy of plasmid pB1. Depending on the particular strain and fermentation conditions, the yield was improved up to eightfold.

1. A nucleic acid molecule encoding a multimeric precursor comprising a protein of interest, wherein the nucleic acid molecule comprises a motif, said motif being repeated at least twice, said motif comprising at least two elements, said at least two elements being:
a) an element encoding a protein of interest,
and
b) an element encoding a cleavage site.

2. A nucleic acid molecule according to claim 1, where the cleavage site is a Kex2 cleavage site.

3. A nucleic acid molecule according to claim 1, wherein the motif additionally comprises a third element being an intervening sequence.

4. A nucleic acid molecule according to claim 1, wherein the protein of interest is selected from the following list: a cytokine, an interleukin, an interferon, a colony stimulating factor, a chemokine, a hormone, a coagulant, an anticoagulant, an antioxidant, an antibody, an engineered immunoglobulin-like molecule, a single chain antibody, a humanised antibody, an immune-costimulatory molecule, an immunomodulatory molecule, a transdominant negative mutant of a target protein, a protein capable of inhibiting a viral, bacterial, or parasitic infection, a structural protein, a fusion protein, an enzyme, a toxin, a conditional toxin, an antigen, a protein capable of inhibiting the inititation or progression of tumours or cancers, a growth factor, a membrane protein, a vasoactive protein, a peptide and a gelatin like protein.

5. A nucleic acid molecule according to claim 4, wherein the protein of interest is a gelatin like protein.

6. A nucleic acid molecule according to claim 4, wherein the protein of interest is an Nanobody®, Avimer®, Anticalin® or Duocalin®.

7. A nucleic acid molecule according to claim 1, wherein:
element a) encodes a protein of interest wherein at least two distinct a) elements are present, each encoding a distinct protein of interest and
element b) encodes a cleavage site wherein at least two b) elements are present, each encoding a cleavage site, so that a cleavage site is present between each protein of interest.

8. A nucleic acid construct or expression vector comprising a nucleic acid molecule as described in claim 1.

9. A cell comprising a nucleic acid construct or expression vector as described in claim 8.

10. A cell according to claim 9, wherein the cell is an eukaryotic cell.

11. A cell according to claim 10, wherein the eukaryotic cell is a yeast cell.

12. A cell according to claim 11, wherein the yeast cell is a Pichia pastoris or a Hansenula polymorpha strain.

13. A cell according to claim 9, comprising, expressing and/or overexpressing a Kex2-like enzyme or an enzyme having Kex2-like processing activity or enhanced Kex2-like processing activity and optionally
further expressing or comprising and/or overexpressing a Kex1-like enzyme or a Kex-1 like having Kex1-like processing activity or enhanced Kex1-like processing activity and/or
further expressing or comprising and/or overexpressing a STE13 gene-like product or a STE13 gene product having the same activity or enhanced activity as a STE13 gene product.

14. Method for the production of a protein of interest using the cell as defined in claim 1.

15. A cell according to claim 11, wherein the yeast cell is a methylotrophic yeast cell.


SEQUENCE LISTING (6 sequences)

SEQ ID NO: 1 - DNA, 2190 bp, Saccharomyces cerevisiae
atgttttacaataggtggctcggaacgtggctagccatgtctgctttaataaggatctcagtatcccttccgtcatctgaggagtacaaagtggcatatgagctgttgccagggttatctgaagtgccagacccttcaaatattccacagatgcatgctggccatattcctttacgttccgaagatgcggatgaacaggatagctctgacttggagtactttttttggaagtttaccaataacgactctaatggcaatgtcgaccgtcccttaattatatggttaaatggtggacccggttgctcttccatggatggtgccttggtcgaatccggcccttttagggtgaattcagacggtaaactttatctaaatgaagggtcctggatatccaaaggcgatcttttatttatcgaccaacctactggtactgggttttctgtcgaacaaaataaagacgaaggtaaaatcgacaaaaacaaatttgacgaagacctagaagatgtgaccaagcattttatggattttctggagaactatttcaagatttttccagaagacctcactaggaaaatcatactatcgggtgaaagttacgctggccaatacataccattctttgccaatgcaattttgaaccataacaaattttcaaagatcgacggggatacatacgacttgaaggcgctattgattggtaacggttggattgaccccaatacacagtccctatcgtaccttccgtttgctatggagaagaaactgattgatgaaagcaaccccaatttcaaacacttaacgaacgcacacgagaattgccagaatctgataaactctgccagtacagatgaggcagcccatttttcgtatcaggaatgtgagaatattttaaaccttctattgtcttataccagggaatcttcgcaaaagggaacagcggattgcttgaacatgtataacttcaatttaaaagatagttatccatcttgtggtatgaattggccgaaagatatttcctttgtcagtaaattcttcagcacacctggtgttattgattcgttgcatcttgattctgataaaattgatcattggaaggaatgcactaatagcgttggaactaaattgtctaatcctatttcaaagccatctatccatttattacctggtctacttgaaagtggaatagagattgtcttgttcaatggtgacaaagacttgatttgtaataacaagggcgtattagatactatagacaatctaaaatggggtggaataaagggatttagcgacgatgctgtttcgttcgattggatccataaatcgaagagtacagacgacagcgaagaatttagcggatacgtgaagtatgatagaaatttgacgtttgttagcgtttataatgcttctcacatggtacccttcgataaaagtttagtgagtagaggcattgtcgatatttactcgaacgatgttatgatcattgacaacaatgggaaaaatgttatgattactactgacgacgatagtgatcaagatgctactactgaaagcggtgataagccaaaagaaaacctcgaagaggaagaacaggaagcgcagaatgaggaaggaaaggaaaaagaaggcaataaagataaagatggcgatgatgataacgacaatgatgacgacgatgaagacgatcacaactccgagggcgacgacgatgatgacgatgacgatgatgaagacgataataatgaaaaacaaagtaatcaaggcctcgaggatagtagacataaatcttcagaatatgaacaagaagaggaagaggtagaagaatttgcagaagagatttcaatgtataaacacaaagcggtagtagttaccatcgttacatttttgatagtggttttaggagtttatgcgtatgatagaagagtgaggagaaaagcgcgtcacacaattttagttgatccaaataatagacaacatgacagtcccaataagacagtttcatgggcagatgatctggaaagcggactcggggcagaagatgatttagaacaagatgagcagttagaaggcggagcacccataagtagcacttctaacaaagcaggatctaaattgaaaactaagaaaaagaagaagtatactagccttccgaacactgaaatcgatgaatcttttgagatgactgatttttaa

SEQ ID NO: 2 - DNA, 2445 bp, Saccharomyces cerevisiae
atgaaagtgaggaaatatattactttatgcttttggtgggccttttcaacatccgctcttgtatcatcacaacaaattccattgaaggaccatacgtcacgacagtattttgctgtagaaagcaatgaaacattatcccgcttggaggaaatgcatccaaattggaaatatgaacatgatgttcgagggctaccaaaccattatgttttttcaaaagagttgctaaaattgggcaaaagatcatcattagaagagttacagggggataacaacgaccacatattatctgtccatgatttattcccgcgtaacgacctatttaagagactaccggtgcctgctccaccaatggactcaagcttgttaccggtaaaagaagctgaggataaactcagcataaatgatccgctttttgagaggcagtggcacttggtcaatccaagttttcctggcagtgatataaatgttcttgatctgtggtacaataatattacaggcgcaggggtcgtggctgccattgttgatgatggccttgactacgaaaatgaagacttgaaggataatttttgcgctgaaggttcttgggatttcaacgacaataccaatttacctaaaccaagattatctgatgactaccatggtacgagatgtgcaggtgaaatagctgccaaaaaaggtaacaatttttgcggtgtcggggtaggttacaacgctaaaatctcaggcataagaatcttatccggtgatatcactacggaagatgaagctgcgtccttgatttatggtctagacgtaaacgatatatattcatgctcatggggtcccgctgatgacggaagacatttacaaggccctagtgacctggtgaaaaaggctttagtaaaaggtgttactgagggaagagattccaaaggagcgatttacgtttttgccagtggaaatggtggaactcgtggtgataattgcaattacgacggctatactaattccatatattctattactattggggctattgatcacaaagatctacatcctccttattccgaaggttgttccgccgtcatggcagtcacgtattcttcaggttcaggcgaatatattcattcgagtgatatcaacggcagatgcagtaatagccacggtggaacgtctgcggctgctccattagctgccggtgtttacactttgttactagaagccaacccaaacctaacttggagagacgtacagtatttatcaatcttgtctgcggtagggttagaaaagaacgctgacggagattggagagatagcgccatggggaagaaatactctcatcgctatggctttggtaaaatcgatgcccataagttaattgaaatgtccaagacctgggagaatgttaacgcacaaacctggttttacctgccaacattgtatgtttcccagtccacaaactccacggaagagacattagaatccgtcataaccatatcagaaaaaagtcttcaagatgctaacttcaagagaattgagcacgtcacggtaactgtagatattgatacagaaattaggggaactacgactgtcgatttaatatcaccagcggggataatttcaaaccttggcgttgtaagaccaagagatgtttcatcagagggattcaaagactggacattcatgtctgtagcacattggggtgagaacggcgtaggtgattggaaaatcaaggttaagacaacagaaaatggacacaggattgacttccacagttggaggctgaagctctttggggaatccattgattcatctaaaacagaaactttcgtctttggaaacgataaagaggaggttgaaccagctgctacagaaagtaccgtatcacaatattctgccagttcaacttctatttccatcagcgctacttctacatcttctatctcaattggtgtggaaacgtcggccattccccaaacgactactgcgagtaccgatcctgattctgatccaaacactcctaaaaaactttcctctcctaggcaagccatgcattattttttaacaatatttttgattggcgccacatttttggtgttatacttcatgttttttatgaaatcaaggagaaggatcagaaggtcaagagcggaaacgtatgaattcgatatcattgatacagactctgagtacgattctactttggacaatggaacttccggaattactgagcccgaagaggttgaggacttcgattttgatttgtccgatgaagaccatcttgcaagtttgtcttcatcagaaaacggtgatgctgaacatacaattgatagtgtactaacaaacgaaaatccatttagtgaccctataaagcaaaagttcccaaatgacgccaacgcagaatctgcttccaataaattacaagaattacagcctgatgttcctccatcttccggacgatcgtga

SEQ ID NO: 3 - protein, 814 aa, Saccharomyces cerevisiae
MKVRKYITLCFWWAFSTSALVSSQQIPLKDHTSRQYFAVESNETLSRLEEMHPNWKYEHDVRGLPNHYVFSKELLKLGKRSSLEELQGDNNDHILSVHDLFPRNDLFKRLPVPAPPMDSSLLPVKEAEDKLSINDPLFERQWHLVNPSFPGSDINVLDLWYNNITGAGVVAAIVDDGLDYENEDLKDNFCAEGSWDFNDNTNLPKPRLSDDYHGTRCAGEIAAKKGNNFCGVGVGYNAKISGIRILSGDITTEDEAASLIYGLDVNDIYSCSWGPADDGRHLQGPSDLVKKALVKGVTEGRDSKGAIYVFASGNGGTRGDNCNYDGYTNSIYSITIGAIDHKDLHPPYSEGCSAVMAVTYSSGSGEYIHSSDINGRCSNSHGGTSAAAPLAAGVYTLLLEANPNLTWRDVQYLSILSAVGLEKNADGDWRDSAMGKKYSHRYGFGKIDAHKLIEMSKTWENVNAQTWFYLPTLYVSQSTNSTEETLESVITISEKSLQDANFKRIEHVTVTVDIDTEIRGTTTVDLISPAGIISNLGVVRPRDVSSEGFKDWTFMSVAHWGENGVGDWKIKVKTTENGHRIDFHSWRLKLFGESIDSSKTETFVFGNDKEEVEPAATESTVSQYSASSTSISISATSTSSISIGVETSAIPQTTTASTDPDSDPNTPKKLSSPRQAMHYFLTIFLIGATFLVLYFMFFMKSRRRIRRSRAETYEFDIIDTDSEYDSTLDNGTSGITEPEEVEDFDFDLSDEDHLASLSSSENGDAEHTIDSVLTNENPFSDPIKQKFPNDANAESASNKLQELQPDVPPSSGRS

SEQ ID NO: 4 - protein, 729 aa, Saccharomyces cerevisiae
MFYNRWLGTWLAMSALIRISVSLPSSEEYKVAYELLPGLSEVPDPSNIPQMHAGHIPLRSEDADEQDSSDLEYFFWKFTNNDSNGNVDRPLIIWLNGGPGCSSMDGALVESGPFRVNSDGKLYLNEGSWISKGDLLFIDQPTGTGFSVEQNKDEGKIDKNKFDEDLEDVTKHFMDFLENYFKIFPEDLTRKIILSGESYAGQYIPFFANAILNHNKFSKIDGDTYDLKALLIGNGWIDPNTQSLSYLPFAMEKKLIDESNPNFKHLTNAHENCQNLINSASTDEAAHFSYQECENILNLLLSYTRESSQKGTADCLNMYNFNLKDSYPSCGMNWPKDISFVSKFFSTPGVIDSLHLDSDKIDHWKECTNSVGTKLSNPISKPSIHLLPGLLESGIEIVLFNGDKDLICNNKGVLDTIDNLKWGGIKGFSDDAVSFDWIHKSKSTDDSEEFSGYVKYDRNLTFVSVYNASHMVPFDKSLVSRGIVDIYSNDVMIIDNNGKNVMITTDDDSDQDATTESGDKPKENLEEEEQEAQNEEGKEKEGNKDKDGDDDNDNDDDDEDDHNSEGDDDDDDDDDEDDNNEKQSNQGLEDSRHKSSEYEQEEEEVEEFAEEISMYKHKAVVVTIVTFLIVVLGVYAYDRRVRRKARHTILVDPNNRQHDSPNKTVSWADDLESGLGAEDDLEQDEQLEGGAPISSTSNKAGSKLKTKKKKKYTSLPNTEIDESFEMTDF

SEQ ID NO: 5 - DNA, 2796 bp, Saccharomyces cerevisiae
atgtctgcttcaactcattcgcataaaaggaagaatagtcatttatttccgcaaaggaaaagttcaaactcttccatggataagccattctttcctaataacgatagtgtagctaacacagatcctcaaagtaacgagaatggacatacaattaacgagattagaccgacagaagctacaatagatgttacagatgttcctcaaaccccctttctccaggaacaatactcaatgcgtcccagacgagaaagcttccagtttaatgatattgaaaaccagcatcacacgcattcctttttcagtgtaaacaaatttaatcgtcgatggggagaatggtctctgcccgagaaaagatcttatgtcttagtctttacattaatagcactatcagttttagttctacttgttattctaataccttcaaaacttctacctaccaaaatcacgcgtcctaagacatctgcaggagattctagtttgggcaaaagatctttctcaattgaaaacgttttgaacggagattttgccatccctgaggatactttccactttattgacccaccacagcgcttattaggccaagactcagatccggggctatattttactacaaaagaaattgatggacatacgaattttattgcaaagcagttatttgatgagacttttgaagtaaaccttgggggcaataggttcctatatgaaggagtagaatttaccgtaagtactgtccaaattaattataaattggataaacttatatttggaactaacttagagtctgagtttagacactcctcgaaggggttttattggatcaaagacttaaacacaggtaatattgaaccgatattacctcccgaaaaatcagatgataattatgaacttggattatcaaaactttcctatgcgcatttttctcctgcatacaattatatctattttgtttacgagaacaatctgtttctgcaacaagtaaatagcggagttgccaagaaggtaacggaagatggctccaaggatatattcaatgccaaaccagactggatatatgaagaagaggtccttgcttctgatcaagcaatatggtgggctccagacgattcgaaggctgtatttgcaagattcaatgacaccagtgttgacgatatccgattaaaccgttacacaaatatgaacgaagcatacctttccgacaccaaaatcaaatacccgaagccagggttccaaaatccacagttcgacttgtttctcgttaacttacagaacggtataatttactcaattaatactggtggtcagaaggattccattctgtataacggaaaatggattagtccagatacattcaggttcgaaatcaccgatagaaactctaagatattagacgtaaaagtgtatgatataccatcatctcaaatgcttactgtgaggaacaccaattcaaacctgttcaacggatggatcgaaaagacgaaggatattttaagcatccccccaaagccagagttgaaaagaatggattatgggtacattgatattcatgcagacagtagagggttcagtcatctcttttattacccaaccgtttttgccaaagagcctatccagttgaccaaagggaactgggaagtgactggaaatggaatagtcggctacgaatatgaaacagatactatattttttactgccaatgagattggtgtcatgtcacaacacctttatagtatcagccttacggattctacaactcagaatactttccaaagtttacaaaacccatcagacaaatacgatttttatgacttcgaattaagttctagtgcgagatatgctatttctaaaaaactggggcctgatacacctattaaagtagctggtccgcttacaagggttttgaacgttgccgaaattcacgatgattcaatcttgcaactgacgaaggatgaaaagttcaaggaaaaaataaagaactacgatttacctattacaagctataaaaccatggtcctagacgatggcgtagaaataaattacattgagatcaaaccagccaatttaaatcctaaaaaaaaatacccgatattagtcaacatttatggtggtccaggatctcaaacatttaccaccaagtcgtctcttgcgtttgaacaagcagttgtttcaggattagatgttatcgtcctacaaatagagcccagaggaactgggggtaaaggttggagttttagatcatgggcccgcgaaaaattaggatattgggaacctagagacattactgaagtcacaaaaaaattcatacaaaggaatagccaacatatagatgaaagcaaaattgctatttggggttggtcatacggcggttttactagcttgaaaaccgtggaattagacaacggcgacacatttaaatacgccatggctgtcgcgccagtaacaaactggacattgtatgattccgtttacacggaaagatatatgaaccaaccatcggaaaaccatgaaggctattttgaagtatccaccatccaaaatttcaaatcattcgaatctttaaagcgtcttttcattgtgcacggaactttcgatgataatgttcacatacaaaatacttttagattagttgatcaactgaatctattgggattgacgaactatgacatgcacatttttccagactctgatcattccatccggtaccacaacgctcaaagaattgtattccaaaaactatactattggttaagggatgcctttgctgaacgctttgacaacactgaagttttgcatttataa

SEQ ID NO: 6 - protein, 931 aa, Saccharomyces cerevisiae
MSASTHSHKRKNSHLFPQRKSSNSSMDKPFFPNNDSVANTDPQSNENGHTINEIRPTEATIDVTDVPQTPFLQEQYSMRPRRESFQFNDIENQHHTHSFFSVNKFNRRWGEWSLPEKRSYVLVFTLIALSVLVLLVILIPSKLLPTKITRPKTSAGDSSLGKRSFSIENVLNGDFAIPEDTFHFIDPPQRLLGQDSDPGLYFTTKEIDGHTNFIAKQLFDETFEVNLGGNRFLYEGVEFTVSTVQINYKLDKLIFGTNLESEFRHSSKGFYWIKDLNTGNIEPILPPEKSDDNYELGLSKLSYAHFSPAYNYIYFVYENNLFLQQVNSGVAKKVTEDGSKDIFNAKPDWIYEEEVLASDQAIWWAPDDSKAVFARFNDTSVDDIRLNRYTNMNEAYLSDTKIKYPKPGFQNPQFDLFLVNLQNGIIYSINTGGQKDSILYNGKWISPDTFRFEITDRNSKILDVKVYDIPSSQMLTVRNTNSNLFNGWIEKTKDILSIPPKPELKRMDYGYIDIHADSRGFSHLFYYPTVFAKEPIQLTKGNWEVTGNGIVGYEYETDTIFFTANEIGVMSQHLYSISLTDSTTQNTFQSLQNPSDKYDFYDFELSSSARYAISKKLGPDTPIKVAGPLTRVLNVAEIHDDSILQLTKDEKFKEKIKNYDLPITSYKTMVLDDGVEINYIEIKPANLNPKKKYPILVNIYGGPGSQTFTTKSSLAFEQAVVSGLDVIVLQIEPRGTGGKGWSFRSWAREKLGYWEPRDITEVTKKFIQRNSQHIDESKIAIWGWSYGGFTSLKTVELDNGDTFKYAMAVAPVTNWTLYDSVYTERYMNQPSENHEGYFEVSTIQNFKSFESLKRLFIVHGTFDDNVHIQNTFRLVDQLNLLGLTNYDMHIFPDSDHSIRYHNAQRIVFQKLYYWLRDAFAERFDNTEVLHL